(19) United States
(12) Patent Application Publication
(10) Pub. No.: US 2009/0001874 A1
(43) Pub. Date: Jan. 1, 2009

(54) ARYLAMINE COMPOUND AND ORGANIC LIGHT EMITTING DEVICE USING IT

(75) Inventor: Feng-Wen Yen, Hsin-Chu (TW)

Correspondence Address: FENG-WEN YEN, 2F, NO. 21, R&D ROAD II, SCIENCE-BASED INDUS. PARK, HSIN-CHU, TW

(73) Assignee: FENG WEN YEN, Hsin-Chu (TW)

(21) Appl. No.: 11/770,729

(22) Filed: Jun. 28, 2007

(51) Int. Cl. H01L 51/50; C07C 211/54

(52) U.S. Cl. 313/504; 564/426; 564/427

(57) ABSTRACT

The present invention discloses an arylamine compound which can be used as hole-injecting or/and hole transporting material or/and emitting host/guest in organic electroluminescence devices is disclosed. The mentioned arylamine compound is represented by the following formula(I) and formula(II):

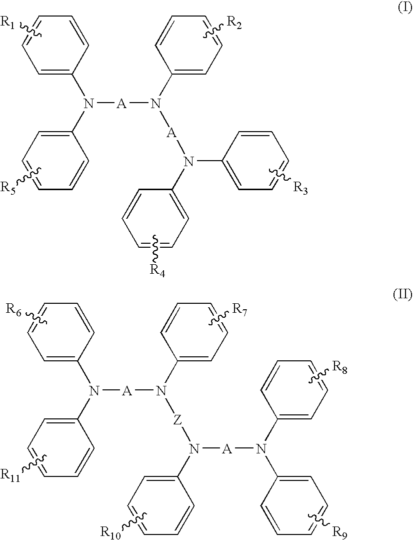

Wherein A is the same or different and is selected from a substituted or unsubstituted anthryl group, a substituted or unsubstituted pyrenyl group, a substituted or unsubstituted perylenyl group, and Z is the same or different and is selected from a substituted or unsubstituted phenyl, a substituted or unsubstituted naphthyl group, a substituted or unsubstituted anthryl group, a substituted or unsubstituted pyrenyl group. R₁ to R₁₁ is selected from a hydrogen atom, a halogen atom, a substituted or unsubstituted amino group, a substituted or unsubstituted alkyl group, a substituted or unsubstituted aryl group, a substituted or unsubstituted aralkyl group, a substituted or unsubstituted heterocyclic group. A organic light emitting device comprising a pair of electrodes consisting of a cathode and an anode, and between the pairs of electrodes comprising at least one layer of arylamine compound of present invention with high efficiency, high luminance and long operation durability and can emit 500 nm˜650 nm of photo-luminescent spectra.

### BACKGROUND OF THE INVENTION

1. Field of the Invention

The present Invention is generally related to arylamine compound and organic light emitting device using the compound. More specifically, the present invention related to arylamine compound having general formula(I) and formula(II), an organic light emitting device employing arylamine compound as hole injection layer or/and hole transporting layer or/and emitting layer of organic light-emitting devices (OLEDs). The OLEDs of present invention can lower power consumption, prolong half-lifetime and increasing efficiency. The arylamine compounds also can emit wide region of fluorescent wavelength from green color to reddish color (550 nm˜650 nm).

2. Description of the Prior Art

Organic light-emitting devices (OLEDs) have received much attention due to their potential applications to flat panel displays. OLEDs are generally composed of functionally divided organic multi-layers, e.g., hole injection layer(HIL), hole transporting layer (HTL), emitting layer(EML) and electron transporting layer (ETL), and so on. Hole injection layer(HIL) and emitting layer (EML) have good charge carrier mobility and excellent operational durability can lower driving voltage and power consumption, increasing efficiency and half-lifetime of OLEDs. Especially the good fluorescent energy transformation from guest to host will increase efficiency and half-lifetime and lower power consumption of OLEDs. Some works had used related arylamine compounds as emitting layer (guest or host) such as “Diaminoanthracene derivatives as High-performance Green Host Electroluminescent Materials”, Chem. Mater., 2002, 14. 3958˜3963, by Chien-Hong Cheng et al., US 2005/0064233 A1 claim amine compounds as guest of emitting layer by Idemitsu Kosan Co., Ltd., US 2006/0082294A1 claim phenylenediamine derivatives as emitting layer or hole transporting layer by Idemitsu Kosan Co., Ltd., US 2006/0113528A1 claim anthracene-based amine as emitting layer by Canon Kabushiki Kaisha. JP 2117971 claim arylamine derivatives as hole transporting material by Sumitomo Chem Co.,Ltd. JP 2873548 also claim triphenylamine compound can be used as hole injection material by Bando Chem Ind., Ltd.

Those arylamine derivatives of prior art emitting blue to green region of fluorescent wavelength, the emitting region needed to be expended to reddish for its more wide application. The arylamine compound still needed corresponding to increase thermal stability and practical operation durability. Especially the half-lifetime and driving voltage needed to be improved for the purpose of industrial practice.

### SUMMARY OF THE INVENTION

In accordance with the present invention, arylamine compounds and their use for hole injection layer or/and hole transporting layer or/and emitting layer of OLEDs are provided. These arylamine compounds can overcome the drawbacks of the prior art.

An object of the present invention is to improve heat-resistant physical characteristic (higher Tₐ) of arylamine compound.

Another object of the present invention is to apply these arylamine compounds for hole injection layer or/and hole transporting layer or/and emitting layer of OLEDs and improve the half-lifetime, lower driving voltage, lower power consumption and increase the efficiency.

Another object of the present invention is to expend the fluorescent wavelength of arylamine compound to reddish region for more wide industrial practice.

The present invention has the economic advantages for industrial practice. Accordingly, the present invention, discloses an arylamine compound which can be used for hole injection layer or/and hole transporting layer or/and emitting layer of OLEDs is disclosed. The mentioned arylamine compound is represented by the following formula(I) and formula(II):

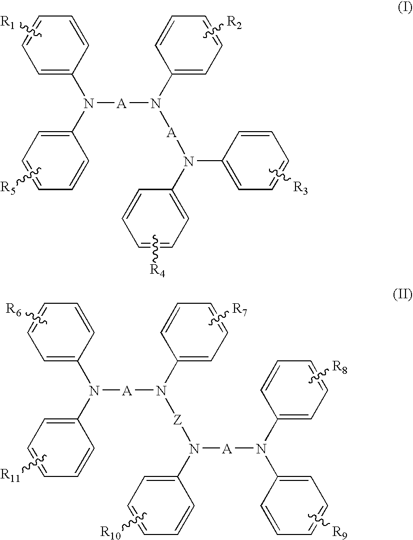

Wherein A is the same or different and is selected from a substituted or unsubstituted anthryl group, a substituted or unsubstituted pyrenyl group, a substituted or unsubstituted perylenyl group, and Z is the same or different and is selected from a substituted or unsubstituted phenyl, a substituted or unsubstituted naphthyl group, a substituted or unsubstituted anthryl group, a substituted or unsubstituted pyrenyl group. R₁ to R₁₁ is selected from a hydrogen atom, a halogen atom, a substituted or unsubstituted amino group, a substituted or unsubstituted alkyl group, a substituted or unsubstituted aryl group, a substituted or unsubstituted aralkyl group, a substituted or unsubstituted heterocyclic group.

### BRIEF DESCRIPTION OF THE DRAWINGS

FIG. 1 show an example of organic light emitting device in the present invention, 1 is transparent electrode, 5 is metal electrode, 2 is hole transporting layer which is deposited onto 1, 3 is emitting layer which is deposited onto 2, 4 is electron transporting layer which is deposited onto 3.

FIG. 2 show an example of organic light emitting device in the present invention, 1 is transparent electrode, 5 is metal electrode, 2 is hole transporting layer which is deposited onto 1, 3 is emitting layer which is deposited onto 2, 4 is electron transporting layer which is deposited onto 3, 6 is hole injection layer inserted on the side of transparent electrode 1 which can improve adhesion between transparent electrode 1 and hole transporting layer 2, or help to increasing hole-injecting capability.

FIG. 3 show the absorption and photo-luminescent spectrum of Example Compound No 2.

FIG. 4 show the absorption and photo-luminescent spectrum of Example Compound No 16.

FIG. 5 is a graph to show thermal degradation temperature (T_{d}) and melting point (Tₘ) of Example Compound No 2.

FIG. 6 is a graph to show thermal degradation temperature (T_{d}) and melting point (Tₘ) of Example Compound No 16.

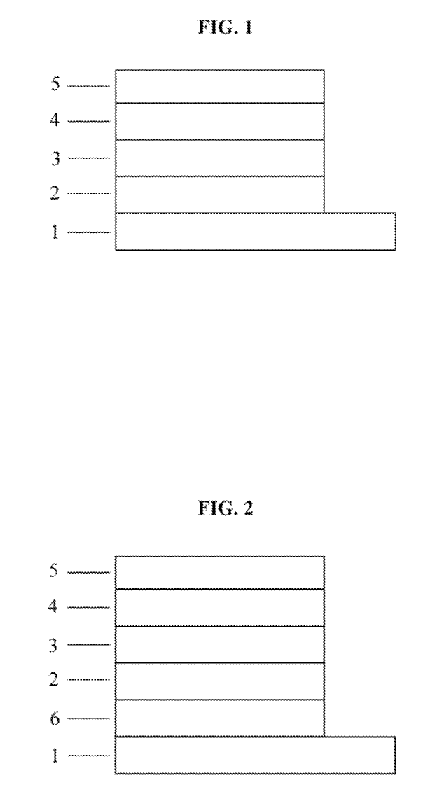

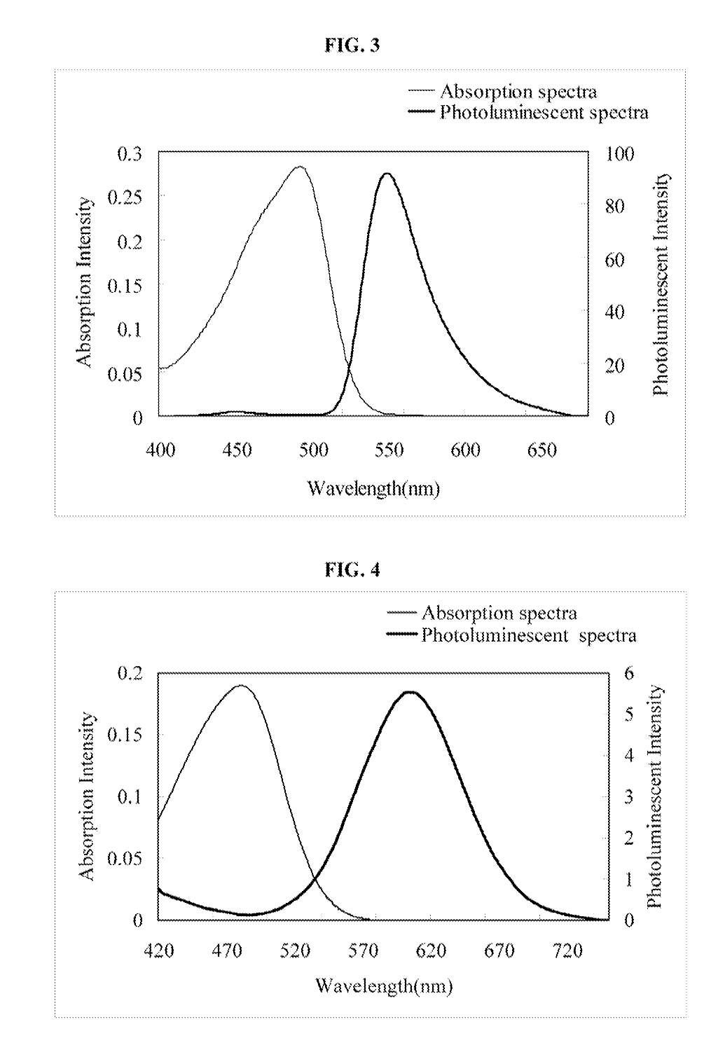

### DESCRIPTION OF THE PREFERRED EMBODIMENTS

What probed into the invention is arylamine compound and organic light emitting device using the compound. Detailed descriptions of the production, structure and elements will be provided in the following to make the invention thoroughly understood. Obviously, the application of the invention is not confined to specific, details familiar to those who are skilled in the art. On the other hand, the common elements and procedures that are known to everyone are not described in details to avoid unnecessary limits of the invention. Some preferred embodiments of the present invention will now be described in greater detail in the following. However, it should be recognized that the present invention can be practiced in a wide range of other embodiments besides those explicitly described, that is, this invention can also be applied extensively to other embodiments, and the scope of the present invention is expressly not limited except as specified in the accompanying claims.

### Definition

The term “thermal degradation temperature (T_{d})” herein refers to the temperature when the weight loss of a heated specimen being 0.5 wt % and “Tₘ” herein refers to Melting point.

In a first embodiment of the present invention, arylamine compound which can be used as hole injection layer or/and hole transporting layer or/and emitting layer of OLEDs is disclosed. The mentioned arylamine compound is represented by following formula(I) and formula(II):

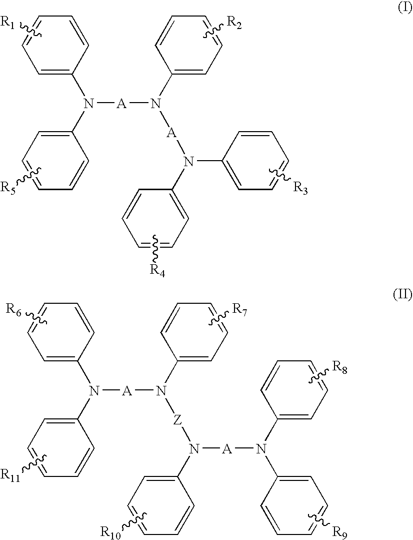

Wherein A is the same or different and is selected from a substituted or unsubstituted anthryl group, a substituted or unsubstituted pyrenyl group, a substituted or unsubstituted perylenyl group, and Z is the same or different and is selected from a substituted or unsubstituted phenyl, a substituted or unsubstituted naphthyl group, a substituted or unsubstituted anthryl group, a substituted or unsubstituted pyrenyl group. R₁ to R₁₁ is selected from a hydrogen atom, a halogen atom, a substituted or unsubstituted amino group, a substituted or unsubstituted alkyl group, a substituted or unsubstituted aryl group, a substituted or unsubstituted aralkyl group, a substituted or unsubstituted heterocyclic group.

In this embodiment some arylamine compounds of the present invention will be shown, but arylamine compound is not limited to the following examples.

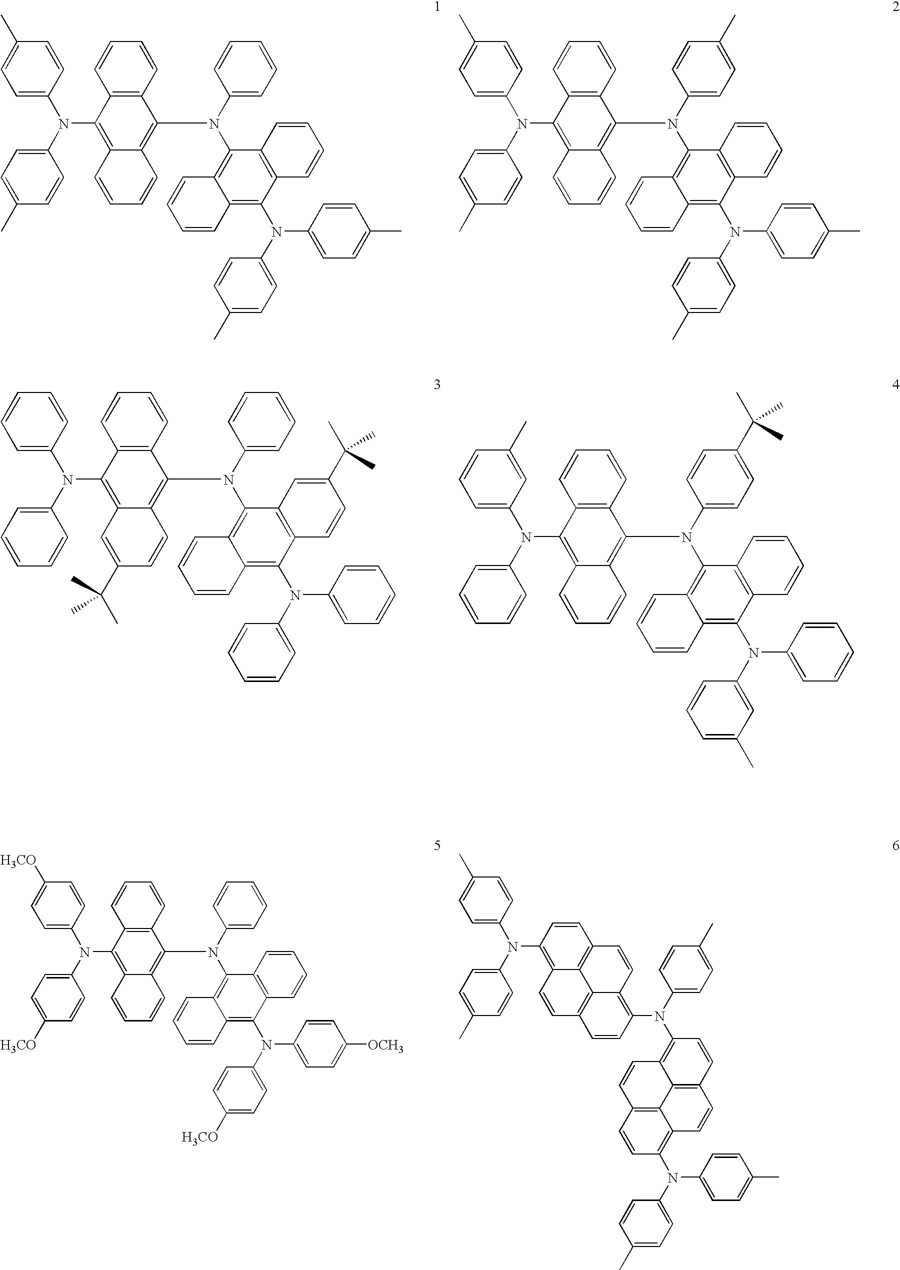

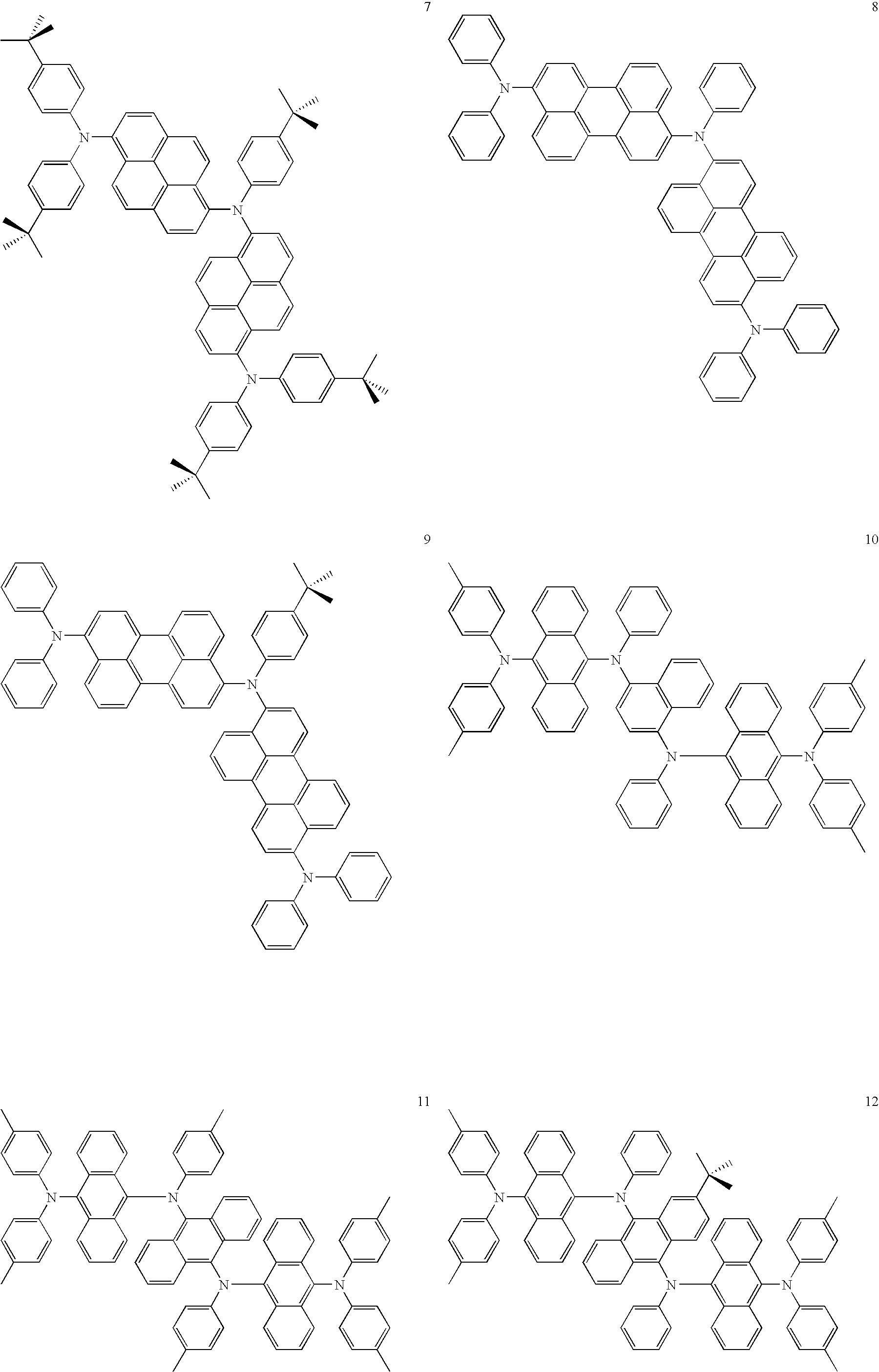

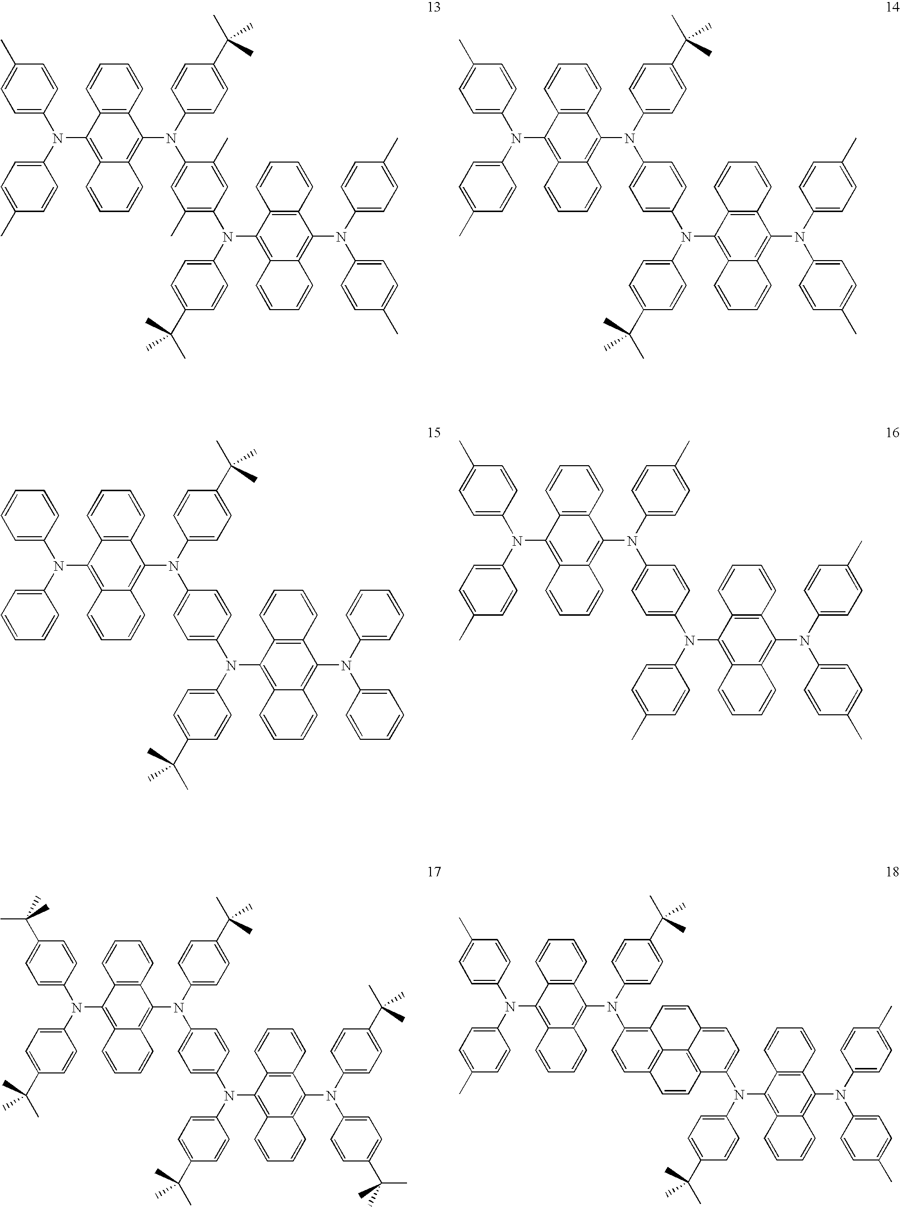

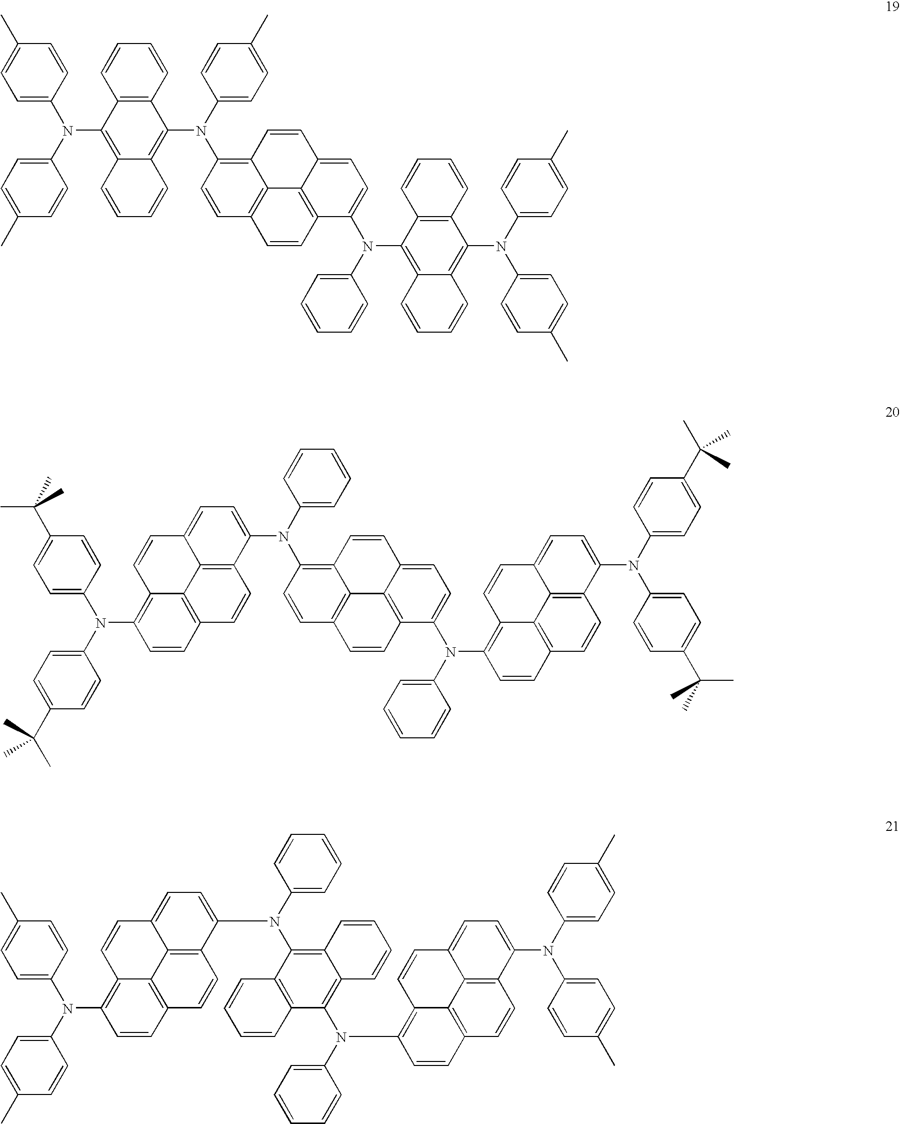

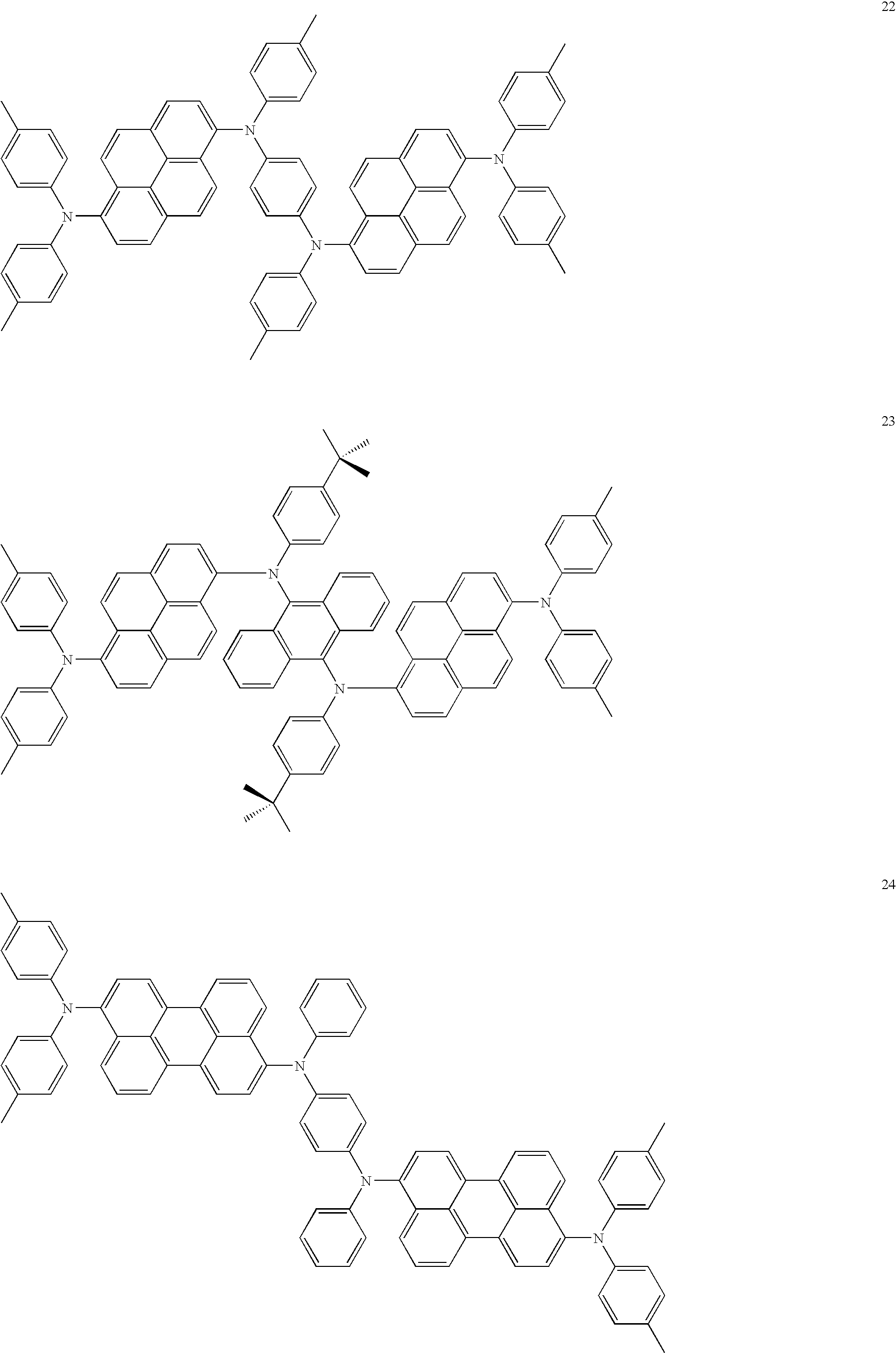

The present invention will be described more specifically based on the following examples.

### EXAMPLE 1

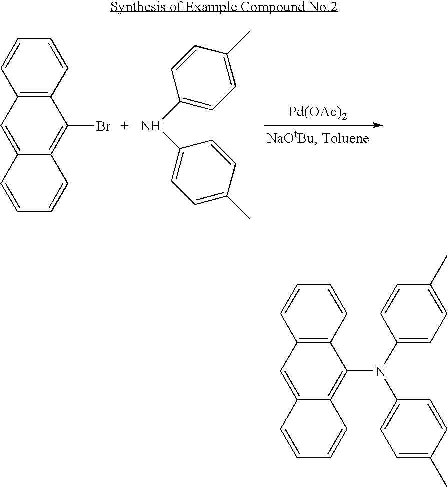

### Synthesis of N,N-di-(p-tolyl)anthracen-10-amine

A mixture of 15 g (58.3 mmol) of 9-bromoanthracene, 13.8 g (70 mmol) of p,p′-ditoylamine, 0.15 g (0.58 mmol) of palladium(II)acetate, 8.4 g (87.5 mmol) of sodium tert-butoxide and 225 ml of dry toluene were refluxed under nitrogen for about 24 h, then cooled to room temperature, the reaction mixture was washed with water and dried with MgSO₄. Most of the solvent was removed under reduced pressure, a yellow precipitate was obtained, the product was dried and got 13 g. Yield=60%.

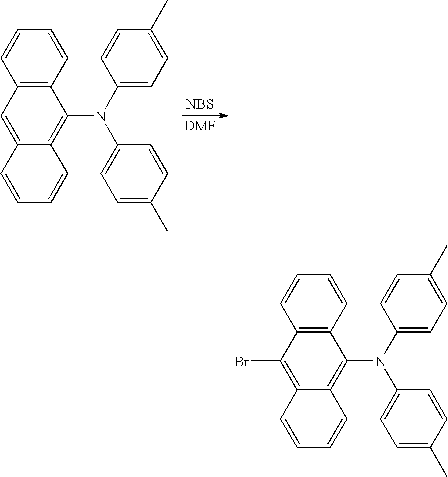

### Synthesis of 9-bromo-N,N-di-(p-tolyl)anthracen-10-amine

A mixture of 13 g (34.8 mmol) of N,N-di-(p-tolyl)anthracen-10-amine and 260 ml of DMF was stirred at room temperature, dropwise addition of a mixture solution of 6.1 g (34.8 mmol) N-Bromosuccnimide and 60 ml of DMF, then stirred overnight at room temperature. The reaction mixture was poured into water, the precipitate was filtered and washed with toluene to give 11.6 g of orange product. Yield=75%.

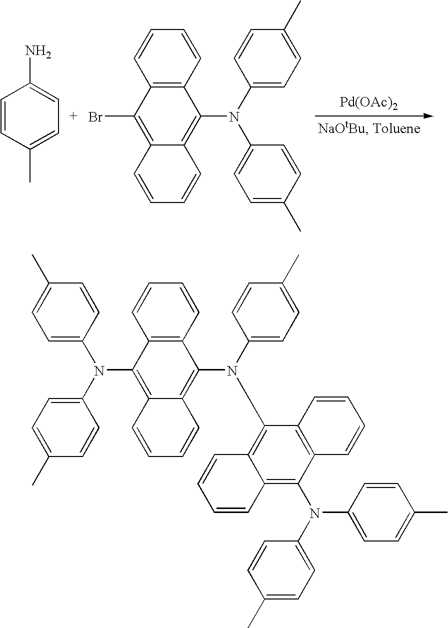

### Synthesis of N¹⁰-(9-(dip-tolylamino)anthracen-10-yl)-N⁹,N⁹,N¹⁰-tri-(p-tolyl)anthracene-9,10-diamine

A mixture of 0.5 g (4.67 mmol) of p-toludine, 4.4 g(9.8 mmol) of 9-bromo-N,N-di-p-tolylanthracen-10-amine, 0.05 g (0.234 mmol) of palladium(II) acetate, 1.34 g (14 mmol) of sodium tert-butoxide and 30 ml of toluene were refluxed under nitrogen for about 24 h, then cooled to room temperature. The precipitate was filtered and washed with methanol to give 2 g of orange product. Yield=50%. Further purification was achieved by sublimation. The product was identified through FAB-MS measurement, m/s=850.

### EXAMPLE 2

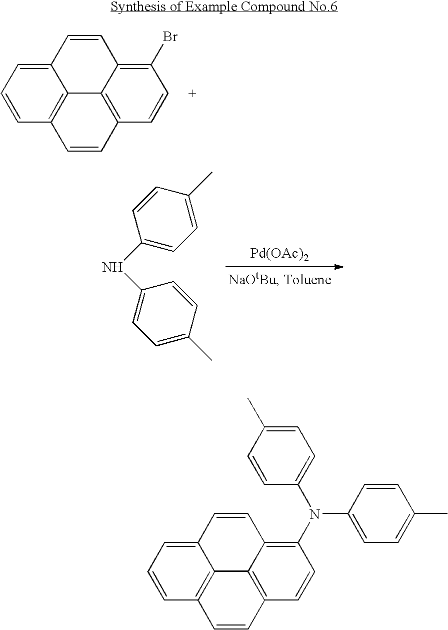

### Synthesis of N,N-di-(p-tolyl)pyren-1-amine

A mixture of 30 g (0.107 mol) of 1-bromopyrene, 25.3 g (0.128 mol,) of p,p′-ditoylamine, 0.24 g (1.07 mmol) of palladium(II) acetate. 15.4 g (0.161 mol) of sodium tert-butoxide and 450 ml of toluene were refluxed under nitrogen for about 24 h, then cooled to room temperature, the reaction mixture was washed with water and dried with MgSO₄. Most of the solvent was removed under reduced pressure, a yellow precipitate was obtained, the product was dried and got 31.5 g. Yield=74%.

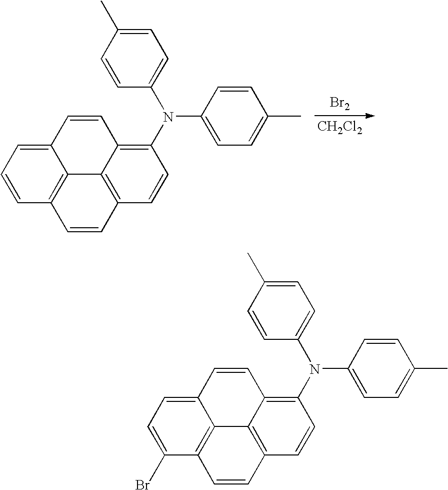

### Synthesis of 6-bromo-N,N-di-(p-tolyl)pyren-1-amine

A mixture of 15 g(37.7 mmol) of N,N-dip-tolylpyren-1-amine and 150 ml of CH₁Cl₁ was stirred at room temperature, dropwise addition of a mixture solution of 6 g(37.7 mmol) N-Bromosuccnimide and 60 ml of CH₂Cl₂, then stirred overnight at room temperature. The reaction mixture was washed with water twice and saturated NaHCO₃ solution, then dried with MgSO₄. The solvent was removed under reduced pressure, the residue was recrystallized from CH₂Cl₂ twice to give 2.5 g of yellow product. Yield=14%.

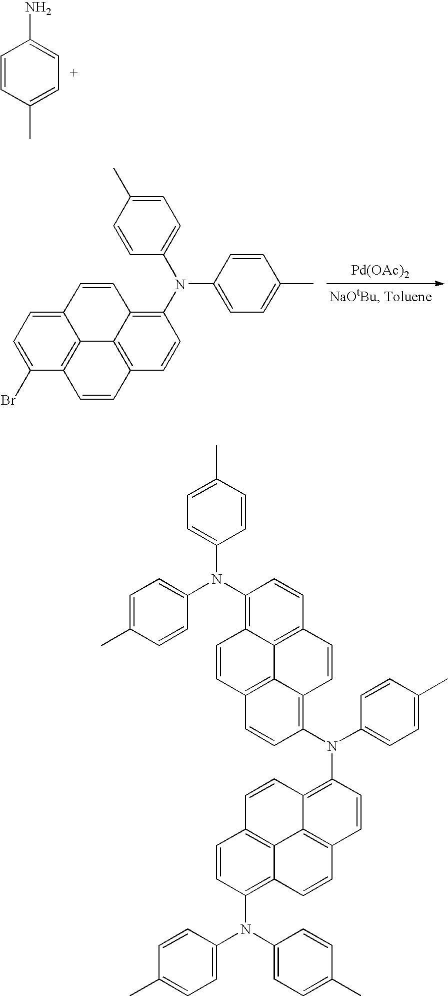

### Synthesis of N¹-(6-(dip-tolylamino)pyren-1-yl)-N¹,N⁶,N⁶-tri-(p-tolyl)pyrene-1,6-diamine

A mixture of 0.5 g (4.67 mmol) of p-toludine. 4.6 g (9.8 mmol) of 6-bromo-N,N-di-(p-tolyl)pyren-1-amine, 0.05 g(0.234 mmol) of palladium(II) acetate, 1.34 g(14 mmol) of sodium tert-butoxide and 30 ml of toluene were refluxed under nitrogen for about 24 h, then cooled to room temperature. The precipitate was filtered and washed with methanol to give 2.3 g of orange product. Yield=55%. Further purification was achieved by sublimation. The product was identified through FAB-MS measurement, m/s=898.

### EXAMPLE 3

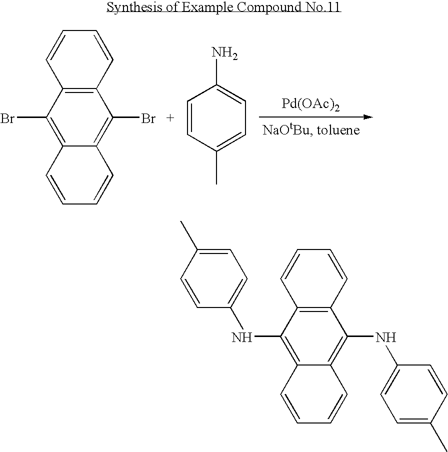

### Synthesis of N⁹N¹⁰-di-(p-tolyl)anthracene-9,10-diamine

A mixture of 10 g (29.7 mmol) of 9,10-dibromoanthracene, 19 g (178.2 mmol) of p-toludine, 0.4 g (1.782 mmol) of palladium(II) acetate, 17.1 g (178.2 mol) of sodium tert-butoxide and 100 ml of toluene were refluxed under nitrogen for about 24 h, then cooled to room temperature. The precipitate was filtered and washed with methanol, the product was purified by silica gel column to give 6 g of orange solid. Yield=52%.

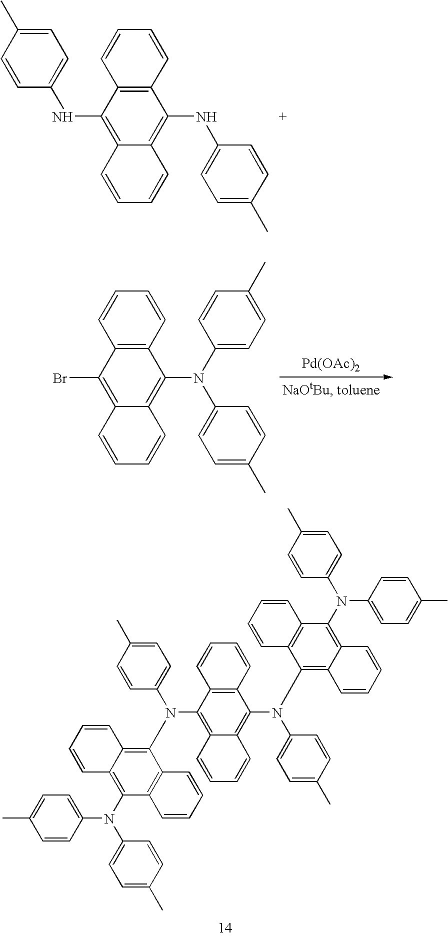

### Synthesis of (N)¹⁰-(4-((9-(di-(p-tolyl)amino)anthracen-10-yl) (p-tolyl)amino)anthracen-10-yl)-N⁹,N⁹,N¹⁰-tri-(p-tolyl)anthracene-9,10-diamine)

A mixture of 0.76 g (1.95 mmol) of N⁹,N¹⁰-di-(p-tolyl)anthracene-9,10-diamine, 1.86 g (4.1 mmol) of 9-bromo-N,N-di-(p-tolyl)anthracen-10-amine, 0.02 g (0.098 mmol) of palladium(II) acetate, 0.56 g (5.85 mmol) of sodium tert-butoxide and 20 ml of toluene were refluxed under nitrogen for about 24 h, then cooled to room temperature. The precipitate was filtered and washed with methanol to give 0.63 g of orange product. Yield=28%. Further purification was achieved by sublimation. The product was identified through TAB-MS measurement, m/s=1131.

### EXAMPLE 4

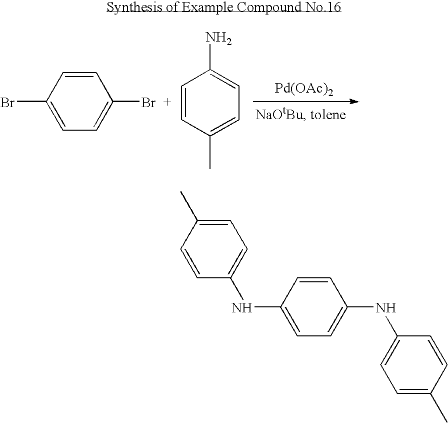

### Synthesis of N¹,N⁴-di-(p-toly)lbenzene-1,4-diamine

A mixture of 10 g (42.4 mmol) of 1,4-dibromobenzene, 22.7 g (0.212 mol) of p-toludine, 0.1 g (0.424 mmol) of palladium(II)acetate, 24.4 g (0.254 mol) of sodium tert-butoxide and 225 ml of toluene were refluxed under nitrogen for about 24 h, then cooled to room temperature, the reaction mixture was washed with water and dried with MgSO₄, the solvent was removed under reduced pressure, the product was purified by silica gel column to give 9 g as white solid. Yield=73%.

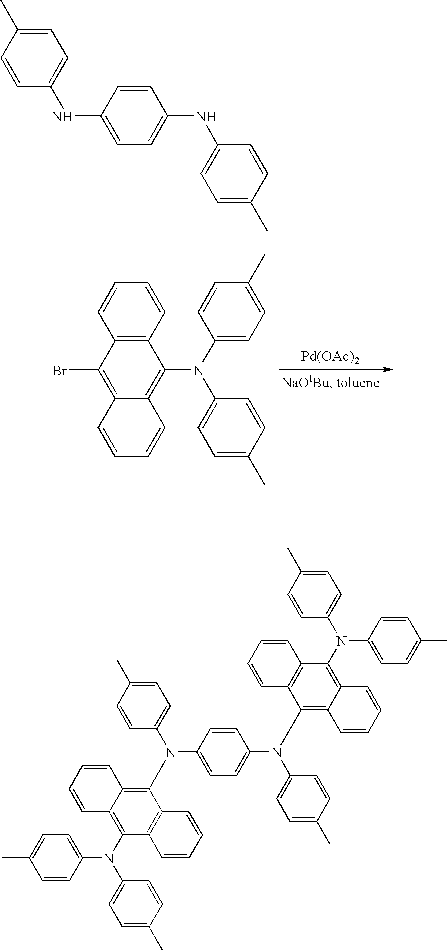

### Synthesis of (N¹⁰-(4-((9-di-(p-toly)lamino)anthracen-10-yl)(p-tolyl)amino)phenyl)-N⁹,N⁹,N¹⁰-tri-(p-tolyl)anthracene-9,10-diamine)

A mixture of 0.7 g (2.42 mmol) of N¹N⁴-di-(p-tolyl)benzene-1,4-diamine, 2.3 g (5.1 mmol) of 9-bromo-N,N-di-(p-tolyl)anthracen-10-amine, 0.025 g (0.121 mmol) of palladium(II) acetate, 0.7 g (7.26 mmol) of sodium tert-butoxide and 20 ml of toluene were refluxed under nitrogen, for about 24 h, then cooled to room temperature. The precipitate was filtered and washed with methanol to give 1.3 g of orange product. Yield=56%. Further purification was achieved by sublimation. The product was identified through FAB-MS measurement, m/s=1031.

### EXAMPLE 5

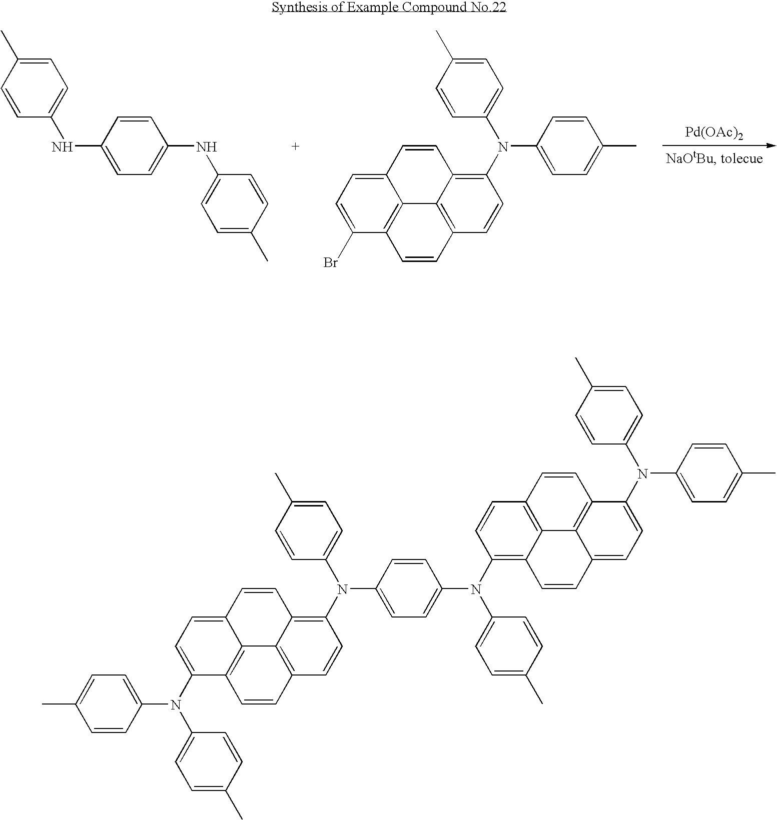

### Synthesis of N¹-(4-((6-(di-(p-tolyl)amino)pyren-1-yl)(p-tolyl)amino)phenyl)-N¹,N⁶,N⁶-tri-(p-tolyl)pyrene-1,6-diamine

A mixture of 0.7 g (2.42 mmol) of N¹,N⁴-di-(p-tolyl)benzene-1,4-diamine, 1.15 g (5.1 mmol) of 6-bromo-N,N-di-(p-tolyl)pyren-1-amine, 0.025 g (0.121 mmol) of palladium(II) acetate, 0.7 g (7.26 mmol) of sodium tert-butoxide and 20 ml of toluene were refluxed under nitrogen for about 24 h, then cooled to room temperature. The precipitate was filtered and washed with methanol to give 1.7 g of orange product. Yield=65%. Further purification was achieved by sublimation. The product was identified through FAB-MS measurement, m/s=1079.

### General Method of Producing OLEDS

ITO-glasses with 15Ω□⁻¹ and 1500 μm in thickness are provided (purchased from Sanyo vacuum, hereinafter ITO substrate) and cleaned in a number of cleaning steps in an ultrasonic bath (e.g. detergent, deionized water). Before vapor deposition of the organic layers, cleaned ITO substrates are further treated by UV and ozone.

These organic layers are applied onto the ITO substrate in order by vapor deposition in a high-vacuum unit. (10⁻⁶ Torr), such, as: resistively heated quartz, boats. The thickness of the respective layer and the vapor deposition rate (0.1˜0.3 nm/sec) are precisely monitored or set with the aid of a quartz-crystal monitor. It is also possible, as described above, for individual layers to consist of more than one compound, i.e. in general a host material doped with a guest material. This is achieved by co-vaporization from two or more sources.

The arylamine compound of present invention represented the above-mentioned general formula(I) and formula(II) can be used as hole injection layer or/and hole transporting layer or/and emitting host/guest of OLEDs. And other general and well-known OLED materials can be collocated with present invention but it is not limited the following examples.

The general and well-known hole injection, material used for OLEDs including; Phthalocyanine, Copper complex (CuPC), 4′,4″-Tris(N-(2-naphthyl)-N-phenyl-amino)triphenyl-amine(2-TNATA), 4,4′,4″-Tris(N-3-methylphenyl-N-phenyl-amino)triphenylamine (m-MTDATA), N,N,N′,N′-Tetrakis(4-methoxyphenyl)benzidine(MeO-TPD) and so on.

N,N′-Bis(naphthalene-1-yl)-N,N′-bis(phenyl)-benzidine (NPB) is most widely used as the hole transporting layer, others such as N,N′-Bis(3-methylphenyl)-N,N′-bis(phenyl)-benzidine(TPD), 2,2′,7,7′-Tetrakis(N,N-diphenylamino)-9,9′-spirobifluorene(Spiro-TAD), 9,9-Bis[4-(N,N-bis-phenyl-4-yl-amino)phenyl]-9H-fluorene(BPAPF), 9,9-Bis[4-(N,N-bis-naphthalen-2-yl-amino)phenyl]-9H-fluorene(NPAPF), 2,2′,7,7′-Tetrakis[N-naphthalenyl(phenyl)-amino]-9,9-spirobifluorene(Spiro-NPB), N,N′-bis(phenanthren-9-yl)-N,N′-bis(phenyl)-benzidine(PAPB). 2,7-Bis[N,N-bis(9,9-spiro-bifluorene-2-yl)-amino]-9,9-spiro-bifluorene(Spiro-5) and so on.

Tris-(8-hydroxyquinoline) aluminum (Alq₃) is most widely used as the electron transporting/light emitting layer in OLEDs for its high thermal stability and good film forming property. It is reported that the thermal degradation, temperature (T_{d}) of Alq₃ is about 303° C. Other electron transporting material such as 2-(4-Biphenylyl)-5-(4-tert-butylphenyl)-1,3,4-oxadiazole(PBD), 2,2′,2″-(1,3,5-Benzinetriyl)-tris(1-phenyl-1-H-benzimidazole)(TPBi), 4,7-Diphenyl-1,10-phenanthroline(BPhen), 3-(4-Biphenylyl)-4-phenyl-5-tert-butylphenyl-1,2,4-triazole(TAZ), 1,3-Bis[2-(4-tert-butylphenyl)-1,3,4-oxadiazo-5-yl]benzene(OXD-7), 1,3-Bis[2-(2,2′-bipyridine-6-yl)-1,3,4-oxadiazo-5-yl]benzene(Bpy-OXD), 1,3-Bis[2-(2,2′-bipyridine-5,5′-dimethyl)-1,3,4-oxadiazo-5-yl]benzene(Bfpy-OXD), 2,7-Bis[2-(2,2′-bipyridine-6-yl)-1,3,4-oxadiazo-5-yl]-9,9-dimethylfluorene(Bpy-FOXD), 2,9-Bis(naphthalen-2-yl)-4,7-diphenyl-1,10-phenanthroline(NBphen), Bis(2-methyl-8-quinolinolate)-4-(phenylphenolato)aluminium(Balq) and so on.

2,3,6,7-Tetrahydro-1,1,7,7-tetramethyl-1H,5H,11H-10-(2-benzo-thiazolyl)quinolizino-[9,9a.1 gh]coumarin(C545T) is widely used as the green guest to co-vaporization with host(Alq₅) for green emissive layer. 4-(Dicyanomethylene)-2-tert-butyl-6-(1,1,7,7-tetramethyljulolidin-4-yl-vinyl)-4H-pyran (DCJTB) is widely used as the reddish guest to co-vaporization with host (Alq₃) for red emissive layer.

Other examples of light emitting materials (host/gust) for fluorescent OLEDs, such as, 9,10-Di(naphth-2-yl)anthracene(AND), 3-Tert-butyl-9,10-di(naphth-2-yl)anthracene(TBADN), 4,4′-Bis(2,2-diphenyl-ethen-1-yl)biphenyl(DPVBi), 2-Methyl-9,10-bis(naphthalen-2-yl)anthracene(MADN), 2,7-Bis[9,9-di(4-methylphenyl)fluorine(TDAF), 2,2′-Di-pyrenyl-9,9-spirobifluorene(2,2′-Spiro-Pye), 2,7-Di-pyrenyl-9,9-spirobifluorene(Spiro-Pye), 1,4-Di(pyren-1-yl)benzene(p-Bpye), 1,3-Di(pyren-1-yl)benzene(m-Bpye), 1,3,5-Tri(pyren-1-yl)benzene(TPB3), 2,2′-Bi(9,10-diphenyl-anthracene(TPBA), 3-(2-Benzothiazolyl)-7-(diethylamino)coumarin(Coumarin 6), N,N′-Dimethyl-quinacridone(DMQA), 9,10-Bis[N,N-di-(p-tolyl)-amino]anthracene(TTPA), 9,10-Bis[phenyl(m-tolyl)-amino]anthracene(TPA), 4,4′-Bis(9-ethyl-3-carbazovinylene)-1,1′-biphenyl(BCzVBi), 2,5,8,11-Tetra-tert-butylperylene(TBPe), 4,4′-Bis[4-(di-p-tolylamino)styryl]biphenyl(DPAVBi), 4-(Di-p-tolylamino-4′-[(di-p-tolylamino)styrl]stilbene(DPAVB), 4,4′-Bis[4-(diphenylamino)styryl]biphenyl(BDAVB), N,N′-Bis(naphthalen-2-yl)-N,N′-bis(phenyl)-tris-(9,9-dimethylfluorenylene)(BNP3FL), N-(4-((E)-2-(6-((E)-4-(diphenylamino)styryl)naphthalen-2-yl)vinyl)phenyl)-N-phenylbenzenamine(N-BDAVBi), 2,7-Bis[4-(diphenylamino)styryl]-9,9-spirobifluorene(Spiro-BDAVBi), 6-Methyl-2-(4-(9-(4-(6-methylbenzo[d]thiazol-2-yl)phenyl)anthracen-10-yl)phenyl)benzo[d]thiazole(DBzA), (5,6,11,12)-Tetraphenylnaphthacene(Rubrene), 2,8-Di-tert-butyl-5,11-bis(4-tert-butylphenyl)-6,12-diphenyltetracene(TBRb) and so on.

Other examples of light emitting materials (host/gust) for phosphorescent OLEDs, such as, Bis(2-methyl-8-quinolinolate)-4-(phenylphenolato)aluminium(BAlq) 1,3-Bis(carbazol-9-yl)benzene(MCP), 1,3,5-Tris(carbazol-9-yl)benzene(TCP), 4,4′,4″-Tris(carbazol-9-yl)triphenylamine(TcTa), 4,4′-Bis(carbazol-9-yl)biphenyl(CBP), 4,4′-Bis(9-carbazolyl)-2,2′-dimethylbiphenyl(CDBP), 2,2′7,7′-Tetrakis(carbazol-9-yl)-9,9′-spirobifluorene(Spiro-CBP), 9,9-Bis[4-(carbazol-9-yl)-phenyl]fluorine(FL-2CBP), 1,4-Bis(triphenylsilyl)benzene(UGH2), 1,3-Bis(triphenylsilyl)benzene(UGH3), Bis(4-N,N-diethylamino-2-methylphenyl)-4-methylphenylmethane(MPMP), Tris(2-phenylpyridine)iridium(III), Ir(ppy)₃, Bis(2-phenylpyridine)(acetylacetonate)iridium(III), Ir(ppy)₂acac) Tris[2-(p-tolyl)pyridine]iridium(III), Ir(mppy)₃, Bis(3,5-difluoro-2-(2-pyridyl)phenyl-(2-carboxypyridyl)iridium III, FIrPic, Bis(2,4-difluorophenylpyridinato)tetrakis(1-pyrazolyl)borate iridium III, FIr6, Tris(dibenzoylmethane)phenanthroline europium(III), Eu(dbm)₃(Phen), Bis(2-benzo[b]thiophen-2-yl-pyridine)(acetylacetonate)iridium(III), Ir(btp)₂(acac), Tris(1-phenylisoquinoline)iridium(III), Ir(piq)₃, Bis(1-phenylisoquinoline)(acetylacetonate)iridium (III), Ir(piq)₂(acac), Bis[1-(9,9-dimethyl-9H-fluoren-2-yl)-isoquinoline](acetylacetonate)iridium(III), Ir(fliq)₂(acac), Bis[3-(9,9-dimethyl-9H-fluoren-2-yl)-isoquinoline](acetylacetonate)iridium(III), Ir(flq)₂(acac), Tris(2-phenylquinoline)iridium(III), Ir(2-phq)₃, Bis(2-phenylquinoline)(acetylacetonate)iridium(III), Ir(2-phq)₂(acac) and so on.

A typical OLED consists of low work function metals, such as Al, Mg, Ca, Li and K, as the cathode by thermal evaporation, and the low work function metals can help electrons injecting the electron transporting layer from cathode, in addition, for reducing the electron injection, barrier and improving the OLED performance, a thin-film electron injecting layer is introduced between the cathode and the electron transporting layer. Conventional materials of electron injecting layer are metal halide or metal oxide with low work function, such as: LiF, MgO, or Li₂O.

On the other hand, after the OLEDs are fabricated, EL spectra and CIE coordination are measured by using a PR650 spectra scan spectrometer. Furthermore, the current/voltage, luminescence/voltage and yield/voltage characteristics are taken with a Keithley 2400 programmable voltage-current source. The above-mentioned apparatuses are operated at room temperature (about 20° C.) and under atmospheric pressure.

### EXAMPLE 6

A reddish organic light emitting device (OLED) was fabricated and tested by above-mentioned general method, structure (see FIG, 2 for contrast) of OLEDs as following: ITO glass/Compound No. 2 (800 Å)/NPB(150 Å)/BAlq+10% Ir(2-phq)₃(350 Å)/Bfpy-OXD(350 Å)/LiF (5 Å)/Al(1600 Å). At a applied voltage of 9V, having an emission luminance of 14100 cd/m² and 1.88 lm/W of power efficiency was got. The CIE color coordinate (x,y)=(0.57, 0.43) and half-life time is 430 hour at an initial luminance of 3000 cd/m².

### EXAMPLE 7

A OLED was fabricated in the same structure and thickness as in Example 6 except that Compound No. 2 was displace with Compound No. 6. At an applied voltage of 9V, having an emission luminance of 16000 cd/m² and 1.69 lm/W of power efficiency was got. The CIE color coordinate (x,y)=(0.56, 0.44) and half-life time is 480 hour at an initial luminance of 3000 cd/m².

### EXAMPLE 8

A OLED was fabricated in the same structure and thickness as in Example 6 except that Compound No. 2 was displace with Compound No. 22. At an applied voltage of 9V, having an emission luminance of 13500 cd/m² and 2.12 lm/W of power efficiency was got. The CIE color coordinate (x,y)=(0.57, 0.43) and half-life time is 390 hour at an initial luminance of 3000 cd/m².

### COMPARATIVE EXAMPLE 1

A OLED was fabricated in the same structure and thickness as in Example 6 except that Compound No. 2 was displace with 2T-NATA(a hole injection material). At an applied voltage of 9V, having an emission luminance of 3400 cd/m² and 2.01 lm/W of power efficiency was got. The CIE color coordinate (x,y)=(0.53, 0,46) and half-life time is 125 hour at an initial luminance of 3000 cd/m².

### EXAMPLE 9

A reddish, organic light emitting device (OLED) was fabricated and tested by above-mentioned general method, structure (see FIG, 1 for contrast) of OLEDs as following: ITO glass/NPB(500 Å)/Alq₃+2% Compound No. 16 (400 Å)/Alq₃(300 Å)/LiF (5 Å)/Al (1600 Å). At an applied voltage of 9V, having an emission luminance of 2260 cd/m² and 1.29 lm/W of power efficiency was got. The CIE color coordinate (x,y)=(0.53, 0.44) and half-life time is 360 hour at an initial luminance of 1000 cd/m².

### EXAMPLE 10

An OLED was fabricated in the same structure and thickness as in Example 9 except that Compound No. 16 was displace with Compound No. 11. At an applied voltage of 9V, having an emission luminance of 1100 cd/m² and 1.25 lm/W of power efficiency was got. The CIE color coordinate (x,y)=(0.50, 0.48) and half-life time is 420 hour at an initial luminance of 1000 cd/m².

### COMPARATIVE EXAMPLE 2

A OLED was fabricated in the same structure and thickness as in Example 6 except that Compound No. 16 was displace with DCJTB (a reddish guest). At an applied voltage of 9V, having an emission luminance of 2400 cd/m² and 1.45 lm/W of power efficiency was got. The CIE color coordinate (x,y)=(0.57, 0.41) and half-life time is 165 hour at an initial luminance of 1000 cd/m².

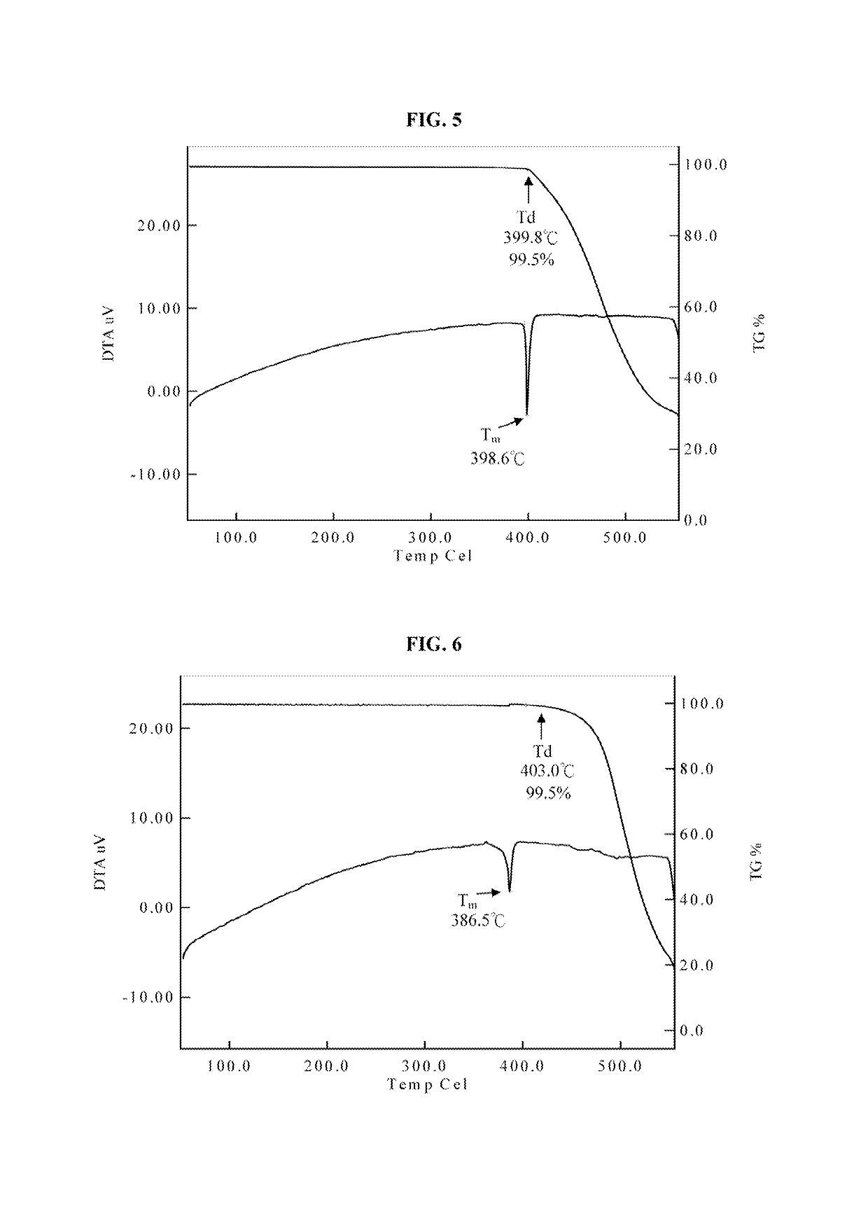

In the above preferred embodiments, we show that arylamine compounds have efficient hole injecting and emitting properties with high thermal stability and practical operation durability. Good performance has also been achieved using the mentioned arylamine compounds for reddish-emitting organic electroluminescent devices. Especially arylamine compounds can be used as red guest of emitting layer because of its photo-luminescence (PL spectrum) over 600 nm of photo-luminescent spectrum (see FIG. 4). Besides arylamine compound of present invention show high Td/Tm (see FIG. 5 and FIG. 6) and keep in solid state under sublimation/deposition process. It is suitable for industrial practice.

To sum up, the present invention discloses a arylamine compound which can be used as hole-injecting or/and hole transporting material or/and emitting host/guest in organic electroluminescence devices is disclosed. The mentioned arylamine compound is represented by the following formula(I) and formula(II):

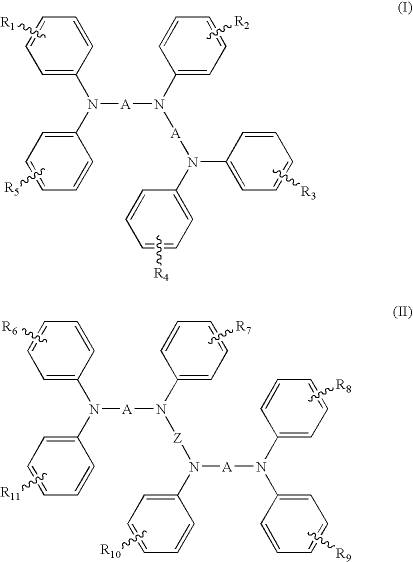

Wherein A is the same or different and is selected from a substituted or unsubstituted anthryl group, a substituted or unsubstituted pyrenyl group, a substituted or unsubstituted perylenyl group, and Z is the same or different and is selected from a substituted or unsubstituted phenyl, a substituted or unsubstituted naphthyl group, a substituted or unsubstituted anthryl group, a substituted or unsubstituted pyrenyl group. R₁ to R₁₁ is selected from a hydrogen atom, a halogen atom, a substituted or unsubstituted amino group, a substituted or unsubstituted alkyl group, a substituted or unsubstituted and group, a substituted or unsubstituted aralkyl group, a substituted or unsubstituted heterocyclic group.

Obviously many modifications and variations are possible in light of the above teachings. It is therefore to be understood that within the scope of the appended claims the present invention can be practiced otherwise than as specifically described herein. Although specific embodiments have been illustrated and described herein, it is obvious to those skilled in the art that many modifications of the present, invention may be made without departing from what is intended to be limited solely by the appended claims.

What is claimed is:

1. A arylamine compound with a general formula(I) and formula(II) as following: 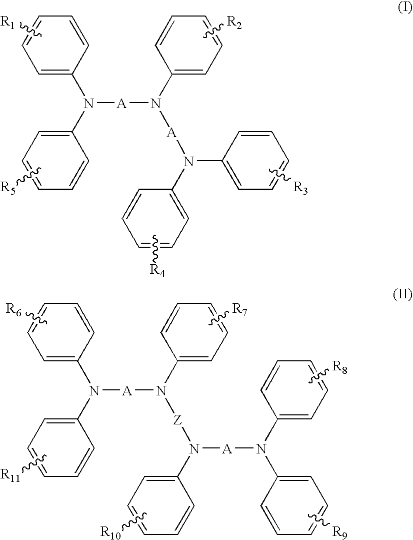
Wherein A is the same or different and is selected from a substituted or unsubstituted anthryl group, a substituted or unsubstituted pyrenyl group, a substituted or unsubstituted perylenyl group, and Z is the same or different and is selected from a substituted or unsubstituted phenyl, a substituted or unsubstituted naphthyl group, a substituted or unsubstituted anthryl group, a substituted or unsubstituted pyrenyl group. R₁ to R₁₃ is selected from a hydrogen atom, a halogen atom, a substituted or unsubstituted amino group, a substituted or unsubstituted alkyl group, a substituted or unsubstituted aryl group, a substituted or unsubstituted aralkyl group, a substituted or unsubstituted heterocyclic group.

2. The compound as claimed in claim 1, wherein the naphthyl group is represented by the general formula (III): 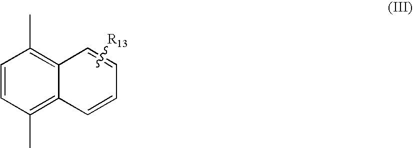
Wherein R₁₃ is selected from a hydrogen atom, a halogen atom, a substituted or unsubstituted amino group, a substituted or unsubstituted alkyl group, a substituted or unsubstituted aryl group, a substituted or unsubstituted aralkyl group, a substituted or unsubstituted heterocyclic group.

3. The compound as claimed in claim 1, wherein the anthryl group is represented by the general formula (IV): 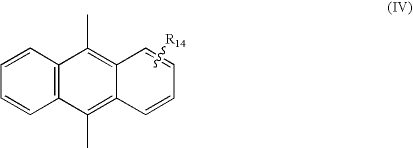
Wherein R₁₄ is selected from a hydrogen atom, a halogen atom, a substituted or unsubstituted amino group, a substituted or unsubstituted alkyl group, a substituted or unsubstituted aryl group, a substituted or unsubstituted aralkyl group, a substituted or unsubstituted heterocyclic group.

4. The compound as claimed in claim 1, wherein the pyrenyl group is represented by the general formula (V): 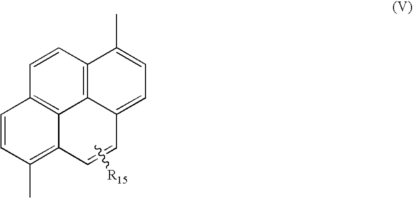
Wherein R₁₅ is selected from a hydrogen atom, a halogen atom, a substituted or unsubstituted amino group, a substituted or unsubstituted alkyl group, a substituted or unsubstituted aryl group, a substituted or unsubstituted aralkyl group, a substituted or unsubstituted heterocyclic group.

5. The compound as claimed in claim 1, wherein the perylenyl group is represented by the general formula (VI): 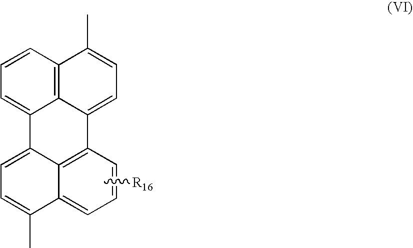
Wherein R₁₆ is selected from a hydrogen atom, a halogen atom, a substituted or unsubstituted amino group, a substituted or unsubstituted alkyl group, a substituted or unsubstituted aryl group, a substituted or unsubstituted aralkyl group, a substituted or unsubstituted heterocyclic group.

6. The compound as claimed in claim 1, wherein R₁ to R₁₁ is selected from alkyl group having 1 to 8 carbon atoms.

7. The compound as claimed in claim 6, wherein R₁ to R₁₁ is independently or dependency methyl group.

8. The compound as claimed in claim 6, wherein R₁ to R₁₁ is independently or dependently tert-butyl group.

9. A organic light emitting device comprising a pair of electrodes consisting of a cathode and an anode, and between the pairs of electrodes comprising at least one layer of compound represented as the following formula(I) and/or formula(II): 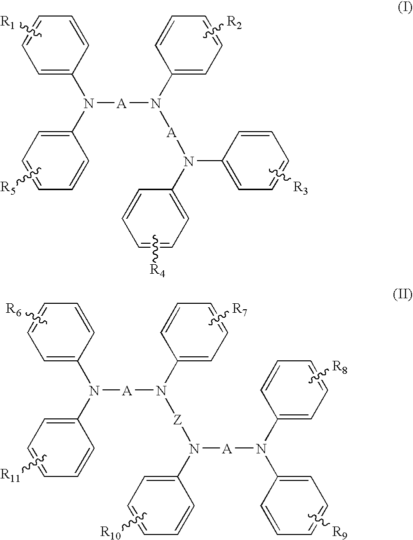
Wherein A is the same or different and is selected from a substituted or unsubstituted anthryl group, a substituted or unsubstituted pyrenyl group, a substituted or unsubstituted perylenyl group, and Z is the same or different and is selected from a substituted or unsubstituted phenyl, a substituted or unsubstituted naphthyl group, a substituted or unsubstituted anthryl group, a substituted or unsubstituted pyrenyl group. R₁ to R₁₁ is selected from a hydrogen atom, a halogen atom, a substituted or unsubstituted amino group, a substituted or unsubstituted alkyl group, a substituted or unsubstituted aryl group, a substituted or unsubstituted aralkyl group, a substituted or unsubstituted heterocyclic group.

10. The organic light emitting device as claimed in claim 9, wherein the naphthyl group is represented by the general formula (III) as above-mentioned.

11. The organic light emitting device as claimed in claim 9, wherein the anthryl group is represented by the general formula (IV) as above-mentioned.

12. The organic light emitting device as claimed in claim 9, wherein the pyrenyl group is represented by the general formula (V) as above-mentioned.

13. The organic light emitting device as claimed in claim 9, wherein the perylenyl group is represented by the general formula (VI) as above-mentioned.

14. The organic light emitting device as claimed in claim 9, wherein R₁ to R₁₁ is selected from alkyl group having 1 to 8 carbon atoms.

15. The organic light emitting device as claimed in claim 14, wherein R₁ to R₁₂ is independently or dependency methyl group.

16. The organic light emitting device as claimed in claim 14, wherein R₁ to R₁₂ is independently or dependency tert-butyl group.

17. The organic light emitting device as claimed in claim 9, arylamine compound represented as the following formula(I) and/or formula(II) as above-mentioned is hole injection layer or/and hole transporting layer or/and emitting layer.

18. The organic light emitting device as claimed in claim 9, arylamine compound represented as the following formula(I) and/or formula(II) as above-mentioned is hole injection layer.

19. The organic light emitting device as claimed in claim 9, arylamine compound represented as the following formula(I) and/or formula(II) as above-mentioned is hole transporting layer.

20. The organic light emitting device as claimed in claim 9, arylamine compound represented as the following formula(I) and/or formula(II) as above-mentioned is emitting layer.

21. The organic light emitting device as claimed in claim 9, arylamine compound represented as the following formula(I) and/or formula(II) as above-mentioned is guest of emitting layer.

22. The organic light emitting device as claimed in claim 9, arylamine compound represented as the following formula(I) and/or formula(II) as above-mentioned can emit fluorescent wavelength from 500 nm to 650 nm.
